(19) United States
(12) Patent Application Publication
(10) Pub. No.: US 2024/0090482 A1
(43) Pub. Date: Mar. 21, 2024

(54) RNAi COMPOSITIONS AND METHODS FOR GENERATION OF SINGLE SEX OFFSPRING

(71) Applicant: Joseph Fenton LAWLER, Spicewood, TX (US)

(72) Inventor: Joseph Fenton LAWLER, Spicewood, TX (US)

(21) Appl. No.: 18/467,026

(22) Filed: Sep. 14, 2023

Related U.S. Application Data
(60) Provisional application No. 63/376,067, filed on Sep. 16, 2022.

(51) Int. Cl. A01K 67/027; A01K 67/02

(52) U.S. Cl. CPC A01K 67/0276; A01K 67/02; A01K 2207/05

(57) ABSTRACT

Described herein are methods and compositions for generating single sex offspring using RNAi approach. In particular, methods and compositions are provided to generate single sex and genetically modified offspring. These techniques can be applied to compassionate animal breeding.

### CROSS REFERENCE

This application claims the benefit of U.S. Provisional Application No. 63/376,067, filed Sep. 16, 2022, which is incorporated herein by reference in its entirety.

### BACKGROUND

In many agricultural applications it is desirable to generate single sex offspring. For example, the products of a mating between two chicken lines optimized for egg laying characteristics are only useful when offspring are female because males cannot lay eggs and breeds used for egg laying are generally not optimized for meat production. As a result, male chicks are separated from females as soon after hatching as possible and culled. Because male and female chicks do not look appreciably different for several days after hatching, specialized methods must be employed to distinguish them. One of the oldest methods involves the use of chicken sexers. These are skilled individuals who are able to determine the sex of a chick before it is obvious to an untrained person. Still, despite their acumen, chicken sexers are not perfect. Moreover, even if chicken sexers were perfectly accurate, they still must wait for the egg to hatch which means eggs harboring males must be carried through the entire incubation process before being identified and culled. This wastes resources on eggs that are not suitable for producing laying hens. Thus, finding new method to generate single sex offspring of animal such as chicken is needed.

### SUMMARY OF THE INVENTION

In some aspects, there are provided non-human vertebrate animals comprising one or more sequence variants of both autosomal genes, and an allosome modified to express one or more RNAi expression cassettes configured to reduce expression of the autosomal gene. In some embodiments, the one or more sequence variants of both autosomal genes do not have sequence identity with the one or more RNAi expression cassettes. In some embodiments, the RNAi expression cassettes are configured to reduce the expression of genes to which they have sequence identity. In some embodiments, the one or more sequence variants of both autosomal genes are resistant to RNAi knock-down. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the autosomal gene is a haploinsufficient gene. In some embodiments, the haploinsufficient gene comprises VEGF, DLL4, or combination thereof. In some embodiments, the haploinsufficient gene is VEGF. In some embodiments, the haploinsufficient gene is DLL4. In some embodiments, the one or more RNAi expression cassettes comprise one or more RNAi sequences operatively linked to one promoter. In some embodiments, the allosome is further modified to overexpress a gene.

In further aspects, there are provided a plurality of non-human vertebrate animals, wherein a first non-human vertebrate animal comprises one or more sequence variants of both autosomal genes, and an allosome modified to express one or more RNAi expression cassettes configured to reduce expression of the autosomal gene. In some embodiments, the one or more sequence variants of both autosomal genes do not have sequence identity with the one or more RNAi expression cassettes, and a second non-human vertebrate animal comprises a wildtype genome. In some embodiments, the allosomes of the first non-human vertebrate animal are heterozygous. In some embodiments, the allosomes of the second non-human vertebrate animal are homozygous. In some embodiments, the RNAi expression cassettes are configured to reduce the expression of genes to which they have sequence identity. In some embodiments, the one or more sequence variants of both autosomal genes in the first non-human vertebrate animal are resistant to RNAi knock-down. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the autosomal gene is a haploinsufficient gene. In some embodiments, the haploinsufficient gene comprises VEGF, DLL4, or a combination thereof. In some embodiments, the haploinsufficient gene is VEGF. In some embodiments, the haploinsufficient gene is DLL4. In some embodiments, the one or more RNAi expression cassettes comprise one or more RNAi sequences operatively linked to one promoter. In some embodiments, the allosome is further modified to overexpress a gene.

In another aspect, there are provided non-human vertebrate animals comprising one or more heterozygous autosomal genes and heterozygous wild-type allosomes, wherein the one or more heterozygous autosomal genes comprises a wildtype genome, and wherein the other of the heterozygous autosomal genes comprises one or more sequence variants of autosomal genes that are resistant to RNAi knock-down. In some embodiments, the autosomal gene is a haploinsufficient gene. In some embodiments, the haploinsufficient gene comprises VEGF, DLL4, or a combination thereof. In some embodiments, the haploinsufficient gene is VEGF. In some embodiments, the haploinsufficient gene is DLL4. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate.

In additional aspects, there are provided methods of producing a single sex offspring of non-human vertebrate animals. In some embodiments, the method comprises: crossing (i) a first non-human vertebrate animal having a first genotype comprising one or more sequence variants of both autosomal genes, and an allosome modified to express one or more RNAi cassettes configured to reduce expression of the autosomal gene, wherein the one or more sequence variants of both autosomal genes do not have sequence identity with the one or more RNAi cassettes with (ii) a second non-human vertebrate animal having a second genotype comprising a wildtype genome with homozygous allosomes; wherein a resulting progeny having a genotype comprising a heterozygous autosomal gene and the allosome modified to express one or more RNAi expression cassettes is not viable; thereby creating a single sex offspring. In some embodiments, the one or more RNAi expression cassettes reduce the expression of genes to which they have sequence identity. In some embodiments, the autosomal gene is a haploinsufficient gene. In some embodiments, the haploinsufficient gene comprises VEGF, DLL4, or a combination thereof. In some embodiments, the haploinsufficient gene is VEGF. In some embodiments, the haploinsufficient gene is DLL4. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the one or more RNAi expression cassettes comprise one or more RNAi sequences operatively linked to one promoter. In some embodiments, the allosome of the first non-human vertebrate animal is further modified to overexpress a gene.

In another aspect, there are provided methods of producing a single sex population of non-human vertebrate animals, comprising the steps of: (a) obtaining (i) a first non-human vertebrate animal having a first genotype comprising one or more sequence variants of an autosomal gene, and an allosome integrated with an RNAi expression cassette comprising the following elements in 5′ to 3′ orientation: a promoter operatively linked thereto a nucleic acid sequence encoding for the expression of one or more RNAi cassettes, wherein the one or more RNAi expression cassettes are configured to reduce the expression of one or more autosomal genes; (b) obtaining (ii) a second non-human vertebrate animal having wildtype genotype; and (c) crossing the first non-human vertebrate animal and the second non-human vertebrate animal, wherein a resulting progeny comprising the allosome integrated with the RNAi expression cassette is not viable; thereby creating a single sex offspring. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the autosomal gene is a haploinsufficient gene. In some embodiments, the haploinsufficient gene comprises VEGF, DLL4, or a combination thereof. In some embodiments, the haploinsufficient gene is VEGF. In some embodiments, the haploinsufficient gene is DLL4. In some embodiments, the promoter is a U6 promoter or a H1 promoter. In some embodiments, the allosome of the first non-human vertebrate animal is further modified to overexpress a gene.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 depicts a genetic cross diagram showing how to generate single sex offspring such as chicken using the methods as described in the present disclosure.

FIG. 2 depicts the Punnett Square of possible genotypic outcomes of offspring from genetic crossing of A*A* and Z¹W with AA and ZZ chicken.

FIG. 3 depicts an example of an RNAi cassette integrated into the allosome. The RNAi cassette expresses short-hairpin RNA (shRNA) to knockdown the haploinsufficient gene on the autosome.

FIG. 4 depicts a genetic cross diagram showing how to generate single sex offspring such as chicken using the RNAi approach with synthetic dosage lethality methods as described in the present disclosure.

FIG. 5 depicts the Punnett Square of possible genotypic outcomes of offspring from genetic crossing of A*A* and Z¹W with AA and ZZ chicken.

### DETAILED DESCRIPTION OF THE INVENTION

In many agricultural applications, generation of single sex offspring, for example, only female hens, is desirable. The products of a mating between two chicken lines optimized for egg laying characteristics are useful when offspring are female because males cannot lay eggs and are generally not optimized for meat production. As a result, male chicks are separated from females as soon after hatching as possible and culled.

According to the statistical report from United States Department of Agriculture (USDA), U.S. egg production totaled 8.67 billion eggs during June 2022, and the total layer hen population in the U.S. on Jul. 1, 2022 is about 366 million (USDA. Chickens and Eggs. July, 2022. ISSN: 1948-9064). Layer hens, however, can lay eggs from 18-19 weeks to 72-78 weeks of age. The layer hen industry, thus, requires the replacement of layer hens annually. Each year, approximately 221.6 million layer hens must be replaced. Assuming an equal sex ratio, this means 523 million layer hen eggs must be hatched of which half will be male. All male chicks will be culled. Each year, up to 300 million male chicks are killed in the U.S., and as many as 7 billion male chicks are culled globally. This presents both animal welfare and ethical issues. In fact, Germany has recently banned the culling of day-old male chicks and Italy plans to follow suit. Companies have also taken notice and recently announced they oppose the practice.

Because male and female chicks do not look appreciably different for several days after hatching, and specialized methods must be employed to distinguish them. One of the oldest methods involves the use of chicken sexers. These are skilled individuals who are able to determine the sex of a chick before it is obvious to an untrained person. Still, despite their acumen, chicken sexers are not perfect, although some can reach greater than 90% accuracy.

Moreover, even if chicken sexers were perfectly accurate, they still must wait for the egg to hatch and grown for several days, which means eggs harboring males must be carried through the entire incubation process before being identified and culled. This wastes resources on eggs which eventually must be culled because male chicks are not suitable for the intended purpose.

Other methods have been developed to separate male and female chicks that rely on feather color (Gohler, D. et al. 2017. Poult Sci. 1; 96(1):1-4). Some methods involve the expression of marker proteins such as green fluorescent protein. Although selectively expressing green fluorescent protein will allow distinguishing between sexes it will also result in genetically modified birds that may not be suitable to customers and are subject to greater regulation (Lee, H. et al. 2019. FASEB J. 33(7):8519-8529).

Methods to allow for selection at the egg stage have also been developed. These methods include detecting minute amounts of estrogen, DNA sequences and other analytes that identify the bird's sex (M-E Krautwald-Junghanns, M. et al. 2018. Poult Sci. 1; 97(3):749-757). These methods, while effective often require additional machinery for implementation and do not escape the fact that male eggs must be incubated to some point.

The present disclosure provides methods and compositions whereby eggs that would otherwise bear male chickens will be suppressed by utilizing an RNAi approach. This approach will improve efficiency in a number of ways. Eggs that would otherwise bear male chicks will be suppressed during embryogenesis, thereby increasing egg hatching capacity significantly. In addition, no screening method would need to be implemented on the eggs nor chicken sexer employed to sort chicks because the laying hen cross from which the eggs are generated usually will not give rise to male offspring.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

As used herein, the terms “allosomes” or “allosome” refer to chromosome that determine gender of an offspring. Allosomes are sometimes referred to as sex chromosomes.

### Animal Genetics and Genotypes

The two categories of chromosomes are autosomes and allosomes (sex chromosomes). Autosomes are other chromosomes that are not allosomes. The allosomes carry the genetic material that determines the gender of an offspring. In mammals, such as humans, cows, or bovines, males are the heterogametic sex which means they have two different sex chromosomes X and Y. The mammalian Y chromosome is a crucial factor for determining gender in mammals. In this case, the female is determined by XX and the male is XY. However, in poultry species and reptiles, such as chickens, females are the heterogametic sex. The allosomes are referred to as Z and W. The female W chromosome in this case is instead an important factor for sex determination. The female chicken has the allosomes ZW while the male chicken has the allosomes ZZ. In male offspring, one of the Z chromosomes is derived from the male parent, while the other Z chromosome is derived from the female parent. In female offspring, the W chromosome is derived from the female parent and the Z chromosome from the male parent.

As used herein, ¹ indication on the chromosomes, e.g., allosome (Z¹ or W¹ in poultry and reptile; X¹ or Y¹ in mammals), refers to the chromosome, e.g., allosome, that is integrated with one or more expression cassettes, e.g., RNAi expression cassette

In some instances, as used herein, * indication on the chromosomes, e.g., autosome such as A*, refers to the chromosome, e.g., autosome, that has a mutated sequence such that it cannot be knocked down by the RNAi, but encodes a functional protein.

Chromosomes and genes come in pairs, and each parent contributes one gene in each pair of genes. If two copies of the genes are the same, the genotype or genetic state is referred to as homozygous. However, if two copies of the genes are different, the genotype in this case is referred to as heterozygous.

In some instances, there are two methods to genetically modify chickens such that a single sex offspring is produced. The first method results in offspring that remains genetically modified in a detectable way, and the other produces chickens that are indistinguishable from wildtype specimens. In either case, the unfertilized egg sold for consumption should be indistinguishable from wildtype as they lack viable cellular material.

CRISPR based approaches can be employed to affect single sex offspring, but because they require the parental birds to express an active CRISPR nuclease, they can result in chromosomal aberrations. These characteristics are undesirable. There is also evidence that birds expressing CRISPR proteins may not grow as well or be as fertile as birds that do not express CRISPR.

In some instances, for genetically modified offspring, the chicken will express the heterologous protein very early in embryogenesis and not thereafter such that its impact, if any is minimized.

This present disclosure provides methods and compositions whereby eggs that would otherwise bear male chickens will be suppressed by utilizing RNAi knockdown of a gene or genes for which haploinsufficiency is lethal. In some instances, the present disclosure provides methods and compositions whereby eggs that would otherwise bear female chickens will be suppressed by utilizing RNAi knockdown of the haploinsufficient gene.

### Haploinsufficiency

In diploid organisms, each gene has two copies, and the expression of protein from both copies contribute to the wildtype phenotype. However, when one copy of the gene is inactivated or deleted or mutated so that the remaining functional copy of the gene is not able to adequately produce wildtype protein to maintain normal function, this can be lethal to a cell. In certain cases, a single copy of a developmentally important genes can be lethal to an organism. This situation is called haploinsufficiency. In some instances, haploinsufficiency comprises a situation in which mutation on protein coding gene can be loss-of-function meaning that the protein can still express but cannot perform or maintain normal function.

As used herein, the terms “haploinsufficient” or “haploinsufficiency” refer to a situation in which the total level of a functional gene product produced by the cell is about half of the normal level and that is not sufficient to permit the cell to function normally. As used herein, the term “haploinsufficient gene” refers to a gene that demonstrates haploinsufficiency in an animal carrying a heterozygous mutation. In some cases, this is lethal to the cell or organism that harbors haploinsufficient gene. Haploinsufficiency leading to embryonic lethality is uncommon with only a few genes having this property. The two best examples are VEGF and DLL4, which are both involved in vasculagenesis. In some cases, a haploinsufficient gene affects the cell or organism via synthetic dosage lethality, where under expression of one gene is made lethal when combined with overexpression of a second gene.

In some instances, the haploinsufficient gene is an essential gene. In some instances, the haploinsufficient gene is an essential gene during embryogenesis.

### RNAi and Haploinsufficiency to Generate Single Sex Offspring

RNA silencing is a process by which mRNA transcript abundance is reduced by either suppressing transcription, which is known as transcriptional gene silencing, or by activating a sequence-specific RNA degradation processes, which is known as posttranscriptional gene silencing or RNA interference (RNAi).

RNAi is a post-transcriptional gene silencing process that is found in many eukaryotic organisms. RNAi is a natural mechanism acting in response to double-stranded RNA (dsRNA) that mediates resistance to both endogenous parasitic and exogenous pathogenic nucleic acids, as well as regulates the expression of protein-coding genes. RNAi has many important and practical applications ranging from therapeutic intervention, functional genomics, animal and agriculture, and other areas. (See Agrawal, N. et al. 2003. MicroBiol Mol Biol Rev; NCBI website on RNAi).

In RNAi mechanism, dsRNA induces sequence-specific gene silencing by targeting mRNA for degradation. RNAi involves multiple steps. First, dsRNA is introduced into the cell, which is then recognized and processed into 21-23 bp small interfering RNAs (siRNA) by Dosha and Dicer. dsRNA may be introduced exogenously and taken up by the cells or by transcription of a dsRNA generating construct within the cell. Next, the siRNAs are incorporated into the RNA-inducing silencing complex (RISC complex). The siRNA is then unwound and the single-stranded RNA guide the RISC complex to the targeted mRNA via hybridizing with the mRNA. Since RISC complex is a nuclease, it induces degradation of the target RNA and complete gene silencing. However, if the siRNA/mRNA duplex contains sequence mismatches, in some cases, the mRNA is not cleaved but translation is inhibited (See Haiyong, H. 2018. Methods Mol Biol; Sontheimer, E. 2005. Nat Rev Mol Cell Biol; NCBI website on RNAi). In some instances, the methods and compositions described herein utilize RNAi approach to generate single sex offspring in non-human animals.

RNAi can be induced in vivo via expression of short hairpin RNA (shRNA) expressed from engineered vector. shRNA can also be synthesized exogenously and introduced into the cell. Once shRNA is present inside the cell, shRNA is processed into siRNA, which can then trigger the RNAi mechanism.

In some instances, the methods and compositions described herein utilize siRNA for RNAi approach to generate single sex offspring in non-human animals. In some instances, the methods and compositions described herein utilized shRNA for RNAi approach to generate single sex offspring in non-human animals.

In some instances, the shRNA is introduced into the cells. In some instances, the shRNA is expressed in the cell via engineered vector containing target sequence for targeted gene. In some instances, the vector encoding shRNA is integrated into a chromosome. In some instances, the vector encoding shRNA is integrated into an allosome. In some instances, the vector encoding shRNA is integrated into an autosome.

In some instances, the siRNA is introduced into the cells. In some instances, the siRNA is expressed in the cell via engineered vector containing target sequence for targeted gene. In some instances, the vector encoding siRNA is integrated into a chromosome. In some instances, the vector encoding siRNA is integrated into an allosome. In some instances, the vector encoding siRNA is integrated into an autosome.

In some instances, the targeted gene is a gene essential for development. In some instances, the targeted gene exhibits developmental haploinsufficiency, which is lethal to the organism. Developmental haploinsufficiency that is lethal to an organism is rare. Examples of genes exhibiting developmentally lethal haploinsufficiency include, but are not limited to, vascular endothelial growth factor (VEGF), delta-like 4 ligand (DLL4). In some embodiments, the haploinsufficient genes are described in, for example, Collins, R. et al. 2022. Cell Press, which is incorporated by reference in its entirety.

In some instances, the RNAi is combined with expression or overexpression of a second gene that interacts with the gene targeted by the RNAi in a phenomenon known as synthetic dosage lethality. In some cases, co-expression of the second gene makes the haploinsufficiency based selection more stringent. For example, if the second gene is overexpressed no effect is observed unless the first gene is under expressed, in which case the combination is lethal. An example of this effect is combining VEGFR1 RNAi to reduce expression of VEGFR1 combined with overexpression of a VEGFR1 mutated to lack a cytoplasmic tail.

### RNAi Expression Cassette

Provided herein are expression cassettes comprising nucleotide sequences encoding a transgene or gene of interest and regulatory sequence to be expressed by a transfected cell. In some instances, the gene of interest comprises component of RNAi knockdown machinery, e.g., siRNA or shRNA. In some instances, the gene of interest comprises a mutated sequence that cannot be knocked down by the RNAi, but encodes a wildtype protein. In some embodiments, one or more expression cassettes are used to generate engineered animal. In some embodiments, the engineered animal includes, but not limited to, cow, mouse, rat, rabbit, guinea pig, chicken, fish, bird, reptile, camelid, bovine, chimpanzee, sheep, goat, and non-human primate.

As used herein, the terms “integration site” or “integrate” refer to the DNA constructs or vectors carrying one or more expression cassette(s) that are integrated into the chromosome in such a way that they are expressed and do not cause significant health issues for animal. In some instances, the one or more expression cassette(s) is integrated into one chromosome. In some instances, the one or more expression cassette(s) is integrated into both chromosomes. In some instances, the chromosome in which the one or more expression cassette(s) is integrated into is an allosome. In some instances, the chromosome in which the one or more expression cassette(s) is integrated into is an autosome.

In some embodiments, the one or more expression cassette(s) is integrated into a chromosome. In some embodiments, the chromosome is an autosome. In some embodiments, the chromosome is an allosome. In some embodiments, the one or more expression cassette(s) is integrated into both chromosomes. In some embodiments, the chromosomes are autosomes.

In some instances, the expression cassette is an RNAi expression cassette. In some instances, the RNAi expression cassette comprises component of RNAi knockdown machinery. In some embodiments, the component of RNAi knockdown machinery encoded by the RNAi expression cassette is siRNA. In some embodiments, the component of RNAi knockdown machinery encoded by the RNAi expression cassette is shRNA. In some embodiments, the shRNA is further processed into siRNA to knockdown a target gene product that has sequence identity, thereby reducing the expression of the gene. In some embodiments, the siRNA encoded by the RNAi expression cassette binds to a gene product, e.g., mRNA, that the siRNA has sequence identity, thereby reducing the expression of the gene. In some embodiments, the shRNA encoded by the RNAi expression cassette binds to the gene product, e.g., mRNA, that the siRNA has sequence identity, thereby reducing the expression of the gene. In some embodiments, the gene that siRNA has sequence identity is a haploinsufficient gene product, e.g., mRNA. In some embodiments, the gene that shRNA has sequence identity is a haploinsufficient gene product, e.g., mRNA.

In some embodiments, the one or more expression cassettes comprise one or more RNAi expression cassettes. In some embodiments, the one or more RNAi expression cassettes carry nucleotide sequence encoding component of RNAi knockdown machinery. In some embodiments, the component of RNAi knockdown machinery comprises siRNA or shRNA. In some embodiments, the one or more RNAi expression cassettes reduce the expression of genes to which they have sequence identity. In some embodiments, the RNAi expression cassette comprises multiple siRNA or shRNA expressed by the same promoter. In some embodiments, the genes are haploinsufficient genes.

As used herein, the terms “promoter” refers to a section of DNA to which proteins, e.g., transcription factors, bind and induce transcription of the adjacent gene located downstream of the promoter. In some instances, promoters can be more active or less active, e.g., driving more transcription or less transcription of the downstream gene either based on their intrinsic strength as a promoter or in response to various signaling events. In some instances, promoters is active at certain times during development, e.g., during embryogenesis. In some instances, promoters is active in certain cell type, e.g., hematopoietic progenitor cells. In some instances, proteins, e.g., transcription factors, can be conditionally recruited to a promoter region to increase transcription or decrease the transcription of the downstream gene. In some instances, wherein the downstream gene encodes for small RNA, e.g., shRNA, as in the RNAi expression cassette, the promoter herein recruits RNA Polymerase III. In this instance, the promoter includes but not limited to U6 promoter or H1 promoter.

In some embodiments, the one or more nucleotide sequences in the RNAi expression cassette further comprise a promoter. In some embodiments, the promoter is normally inactive in the adult non-human vertebrate animal. In some embodiments, the promoter is active during embryogenesis. In some embodiments, the promoter is active during embryogenesis and is silent or suppressed after embryogenesis. In some embodiments, the promoter is activated by a transcription factor.

In some embodiments, the promoter is normally active in the adult non-human vertebrate animal. In some embodiments, the promoter is inactive during embryogenesis. In some embodiments, the promoter is active in a wide range of cell types. In some embodiments, the promoter is active in a specific cell type.

In some embodiments, the promoter in RNAi expression cassette recruits RNA Polymerase III. In some embodiments, the promoter in the RNAi expression cassette is a U6 promoter. In some embodiments, the promoter in the RNAi expression cassette is a H1 promoter.

In some embodiments, the one or more RNAi expression cassettes carry nucleotide sequence encoding component of RNAi knockdown machinery. In some embodiments, the component of RNAi knockdown machinery comprises siRNA or shRNA. In some embodiments, the siRNA or shRNA has sequence identity to untranslated regions of the mRNA. In some embodiments, the siRNA or shRNA has sequence identity to 5′ UTR of the mRNA. In some embodiments, the siRNA or shRNA has sequence identity to 3′ UTR regions of the mRNA. In some embodiments, the siRNA or shRNA has sequence identity to intron regions of the mRNA. In some embodiments, the siRNA or shRNA has sequence identity to translated regions of the mRNA. In some embodiments, the siRNA or shRNA has sequence identity to exon regions of the mRNA. In some embodiments, the mRNA is encoded from an essential gene. In some embodiments, the essential gene is a haploinsufficient gene.

In some embodiments, the one or more RNAi expression cassettes are introduced in combination with an expression cassette which overexpresses a gene that interacts with the target of the RNAi in synthetic dose lethality. In some embodiments, overexpression of the gene does not have any significant effect on the organism without combination of the knockdown of the RNAi. In some embodiments, the overexpressed gene is found on the same chromosome as the one or more RNAi expression cassettes. In some embodiments, the gene that overexpresses the gene is a constitutive promoter. In some embodiments, the promoter is active in the adult non-human vertebrate animal. In some embodiments, the promoter is inactive during embryogenesis. In some embodiments, the promoter is cell type specific. In some embodiments, the overexpressed gene has a modification compared with a wildtype version of the gene. In some embodiments, the modification is a single base substitution, an insertion, or a deletion.

Delivery of the DNA constructs carrying one or more expression cassette(s) to generate engineered animal, e.g., chicken, is performed by viral transfection system, e.g., lentiviral based system. Alternatively, non-viral method is utilized. The non-viral method is based on genetically modified embryonic cells carrying DNA construct to be transferred into the recipient embryo, thereby generating transgenic/engineered animal, e.g., chicken (see Bednarczyk, M. et al. 2018. 59:81-89).

In some embodiments, the method to generate engineered animal, e.g., chicken, comprises viral transfection system. In some embodiments, the viral transfection system is a lentiviral based system. In various embodiments, the method to generate engineered animal, e.g., chicken, comprises non-viral method, e.g., electroporation or lipofection to transfer DNA construct into the targeted cell. In some cases, CRISPR is used to introduce a transgene into the targeted cell.

### Mutated Haploinsufficient Gene Sequences

Engineered animal, e.g., chicken, with RNAi expression cassette targeting haploinsufficient gene product, e.g., mRNA, is also modified so that the RNAi knockdown machinery, e.g., siRNA or shRNA, cannot occur in this engineered animal. This engineered animal is used for breeding with a wildtype animal to generate single sex offspring.

In some instances, the engineered animal, e.g., chicken, comprises a modified genotype with one or more sequence variants of both autosomal genes mutated to not have sequence identity with one ore more RNAi expression cassette. In some instances, the engineered animal, e.g., chicken, further comprises an allosome modified to express one or more RNAi expression cassettes configured to reduce expression of the autosomal gene. In some instances, the engineered animal is a non-human vertebrate animal, including but not limited to cow, mouse, rat, rabbit, guinea pig, chicken, fish, bird, reptile, camelid, bovine, chimpanzee, sheep, goat, and non-human primate.

In some instances, the engineered animal, e.g., chicken, comprises an allosome modified to express one or more RNAi expression cassettes configured to reduce expression of the autosomal gene. In some instances, the engineered animal, e.g., chicken further comprises a modified genotype with one or more sequence variants of both autosomal genes mutated to not have sequence identity with one or more RNAi expression cassette. In some instances, the engineered animal is a non-human vertebrate animal, including but not limited to cow, mouse, rat, rabbit, guinea pig, chicken, fish, bird, reptile, camelid, bovine, chimpanzee, sheep, goat, and non-human primate.

In some instances, the mutated haploinsufficient gene comprises a mutated sequence that cannot be knocked down by the RNAi, but encodes a wildtype protein. In some instances, the mutated sequence is located in introns. In some instances, the mutated sequence is located in 5′UTR. In some instances, the mutated sequence is located in 3′UTR. In some instances, the mutated sequence is not located in exons. In some instances, the mutated sequence that does not affect wildtype protein expression. In some instances, the mutated sequence encodes a haploinsufficient gene product. In some instances, the mutated sequence is a naturally occurring variants. In some instances, the mutated sequence is generated via genetic engineered tools, e.g., CRISPR-Cas9 system, zinc-finger nucleases (ZFNs), or chemically mutagenesis. Other engineering tools to mutate nucleotide sequence can be applied to this present disclosure. Further, this RNAi and haploinsufficiency for generation of single sex offspring can be applied to other animal, all of which are compatible with methods of the present disclosure and contemplated herein. Examples of animal include, but not limited to cow, mouse, rat, rabbit, guinea pig, chicken, fish, bird, reptile, camelid, bovine, chimpanzee, sheep, goat, and non-human primate.

### Animals for Generation of Single Sex Offspring Via RNAi and Haploinsufficiency

Provided herein are non-human vertebrate animals comprising one or more sequence variants of both autosomal genes, and an allosome modified to express one or more RNAi expression cassettes configured to reduce expression of the autosomal gene. In some embodiments, the one or more sequence variants of both autosomal genes do not have sequence identity with the one or more RNAi expression cassettes. In some embodiments, the RNAi expression cassettes are configured to reduce the expression of genes to which they have sequence identity. In some embodiments, the one or more sequence variants of both autosomal genes are resistant to RNAi knock-down. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the autosomal gene is a haploinsufficient gene. In some embodiments, the haploinsufficient gene comprises VEGF, DLL4, or combination thereof. In some embodiments, the haploinsufficient gene is VEGF. In some embodiments, the haploinsufficient gene is DLL4. In some embodiments, the one or more RNAi expression cassettes comprise one or more RNAi sequences operatively linked to one promoter. In some embodiments, the allosome is further modified to overexpress a gene.

Additionally provided herein are pluralities of non-human vertebrate animals, wherein a first non-human vertebrate animal comprises one or more sequence variants of both autosomal genes, and an allosome modified to express one or more RNAi expression cassettes configured to reduce expression of the autosomal gene. In some embodiments, the one or more sequence variants of both autosomal genes do not have sequence identity with the one or more RNAi expression cassettes, and a second non-human vertebrate animal comprises a wildtype genome. In some embodiments, the allosomes of the first non-human vertebrate animal are heterozygous. In some embodiments, the allosomes of the second non-human vertebrate animal are homozygous. In some embodiments, the RNAi expression cassettes are configured to reduce the expression of genes to which they have sequence identity. In some embodiments, the one or more sequence variants of both autosomal genes in the first non-human vertebrate animal are resistant to RNAi knock-down. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the autosomal gene is a haploinsufficient gene. In some embodiments, the haploinsufficient gene comprises VEGF, DLL4, or a combination thereof. In some embodiments, the haploinsufficient gene is VEGF. In some embodiments, the haploinsufficient gene is DLL4. In some embodiments, the one or more RNAi expression cassettes comprise one or more RNAi sequences operatively linked to one promoter. In some embodiments, the allosome is further modified to overexpress a gene.

### Poultry

Engineered poultry, e.g., chickens, are generated for generation of single sex offspring, e.g., female layer hens. In some instances, a female chicken in the first generation is engineered to harbor one or more expression cassette(s) encoding the dsRNA needed to direct the RNAi knockdown machinery, e.g., siRNA or shRNA, targeting an autosomal gene. In some instances, the autosomal gene targeted by RNAi is a haploinsufficient gene. This RNAi expression cassette is integrated into the female chicken chromosome. In some instances, the one or more RNAi expression cassette(s) is integrated into the Z allosome (called Z¹). In some instances, an expression cassette that overexpresses a gene that causes synthetic dosage lethality with the RNAi expression cassette is also integrated into the Z allosome (called Z¹). In this instance, both copies of the targeted autosomal genes of the engineered female chicken are modified to be resistant to RNAi knockdown, but express a wildtype protein. In other words, the gene of interest, e.g., haploinsufficient gene, comprises a mutated sequence that cannot be knocked down by RNAi, but encodes a wildtype protein. In this instance, the genotype of the engineered female chicken is A*A* and Z¹W, wherein A* indicates mutated sequence that cannot be knocked down by RNAi, but encodes wildtype protein. This chicken is phenotypically normal. In this instance, the genotype of the male chicken used for crossing with the engineered female chicken to generate single sex offspring, e.g., female chicken, is wildtype AA and ZZ. Further, if the engineered female chicken A*A* Z¹W is crossed with a A*A* Z¹Z¹ male, the cross will result in male and female chickens allowing maintenance of the flock.

In some instances, the targeted autosomal gene is a gene essential for development. In some instances, the targeted gene is a haploinsufficient gene, which is lethal to the organism. Examples of haploinsufficient gene include, but not limited to, vascular endothelial growth factor (VEGF) or delta-like 4 ligand (DLL4). In some embodiments, the haploinsufficient genes are described in, for example, Collins, R. et al. 2022. Cell Press, which is incorporated by reference in its entirety.

In order to generate the offspring generation of chicken from the engineered female chicken and wildtype male chicken, A*A* and Z¹W engineered female chicken is crossed with wildtype AA and ZZ male chicken. This will generate viable A*A and ZW female offspring chicken but not viable male offspring because A*A and Z¹Z male offspring express components of the RNAi knockdown machinery, e.g., siRNA or shRNA, which reduce the expression of wildtype A gene product. Thus, male chicken is not viable. Generation of single sex offspring, e.g., female chicken, is achieved via RNAi and developmentally lethal haploinsufficiency. This RNAi and developmentally lethal haploinsufficiency for generation of single sex offspring can be applied to other animals, all of which are compatible with methods of the present disclosure and contemplated herein.

### Mammals

Engineered mammals, e.g., cows, are generated for generation of single sex offspring, e.g., female cows. In some instances, a male cow in the first generation is engineered to harbor one or more expression cassette(s) encoding component of RNAi knockdown machinery, e.g., siRNA or shRNA, targeting an autosomal gene. In some instances, the autosomal gene targeted by RNAi is a haploinsufficient gene. This RNAi expression cassette is integrated into the female chicken chromosome. In some instances, the one or more RNAi expression cassette(s) is integrated into the Y allosome (called Y¹). In some instances, an expression cassette that overexpresses a gene that causes synthetic dosage lethality with the RNAi expression cassette is also integrated into the Z allosome (called Z¹). In this instance, both copies of the targeted autosomal genes of the engineered male cow are modified to be resistant to RNAi knockdown, but express a wildtype protein. In other words, the gene of interest, e.g., haploinsufficient gene, comprises a mutated sequence that cannot be knocked down by RNAi, but encodes a wildtype protein. In this instance, the genotype of the engineered male cow is A*A* and XY¹, wherein A* indicates mutated sequence that cannot be knocked down by RNAi. In this instance, the genotype of the female cow used for crossing with the engineered male cow to generate single sex offspring, e.g., female cows, is wildtype AA and XX.

In some instances, the targeted autosomal gene is a gene essential for development. In some instances, the targeted gene is haploinsufficient gene, which is lethal to the organism. Examples of haploinsufficient gene include, but not limited to, vascular endothelial growth factor (VEGF), delta-like 4 ligand (DLL4). In some embodiments, the haploinsufficient genes are described in, for example, Collins, R. et al. 2022. Cell Press, which is incorporated by reference in its entirety.

In order to generate the offspring generation of cows from the engineered male cow and wildtype female cow, A*A* and XY¹ engineered male cow is crossed with wildtype AA and XX female cow. This will generate viable A*A and XX female offspring cow because A*A and XY¹ male offspring expresses component of RNAi knockdown machinery, e.g., siRNA or shRNA, which reduce the expression of wildtype A gene product. Thus, male cows are not viable. Generation of single sex offspring, e.g., female cow, is achieved via RNAi and haploinsufficiency. This RNAi and haploinsufficiency for generation of single sex offspring can be applied to other animals, all of which are compatible with methods of the present disclosure and contemplated herein. Examples of animal include, but not limited to mammals, e.g., cow, mouse, rat, rabbit, guinea pig, bovine, chimpanzee, sheep, goat, and non-human primate.

### Methods for Generation of Single Sex Offspring Via RNAi and Haploinsufficiency

By crossing the engineered animal as described in the present disclosure, generation of single sex offspring can be achieved. In one aspect, the present disclosure provides methods of producing a single sex offspring of non-human vertebrate animals. In some embodiments, the method comprises crossing a first non-human vertebrate animal having a first genotype comprising one or more sequence variants of both autosomal genes, and an allosome modified to express one or more RNAi cassettes configured to reduce expression of the autosomal gene, wherein the one or more sequence variants of both autosomal genes do not have sequence identity with the one or more RNAi cassettes with a second non-human vertebrate animal having a second genotype comprising a wildtype genome with homozygous allosomes; wherein a resulting progeny having a genotype comprising a heterozygous autosomal gene and the allosome modified to express one or more RNAi expression cassettes is not viable; thereby creating a single sex offspring. In some embodiments, the one or more RNAi expression cassettes reduce the expression of genes to which they have sequence identity. In some embodiments, the autosomal gene is a haploinsufficient gene. In some embodiments, the haploinsufficient gene comprises VEGF, DLL4, or a combination thereof. In some embodiments, the haploinsufficient gene is VEGF. In some embodiments, the haploinsufficient gene is DLL4. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the one or more RNAi expression cassettes comprise one or more RNAi sequences operatively linked to one promoter. In some embodiments, the allosome of the first non-human vertebrate animal is further modified to overexpress a gene.

In another aspect, there are provided methods of producing a single sex population of non-human vertebrate animals comprising the steps of: obtaining a first non-human vertebrate animal having a first genotype comprising one or more sequence variants of an autosomal gene, and an allosome integrated with an RNAi expression cassette comprising the following elements in 5′ to 3′ orientation: a promoter operatively linked thereto a nucleic acid sequence encoding for the expression of one or more RNAi cassettes, wherein the one or more RNAi expression cassettes are configured to reduce the expression of one or more autosomal genes; obtaining a second non-human vertebrate animal having wildtype genotype; and crossing the first non-human vertebrate animal and the second non-human vertebrate animal, wherein a resulting progeny comprising the allosome integrated with the RNAi expression cassette is not viable; thereby creating a single sex offspring. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the autosomal gene is a haploinsufficient gene. The haploinsufficient gene comprises VEGF, DLL4, or a combination thereof. In some embodiments, the haploinsufficient gene is VEGF. In some embodiments, the haploinsufficient gene is DLL4. In some embodiments, the promoter is a U6 promoter or a H1 promoter. In some embodiments, the allosome of the first non-human vertebrate animal is further modified to overexpress a gene.

### Single Sex Offspring Generated Via RNAi and Haploinsufficiency

In one aspect, the present disclosure provides non-human vertebrate animals comprising one or more heterozygous autosomal genes and heterozygous wild-type allosomes. In some embodiments, the one or more heterozygous autosomal genes comprises a wildtype genome, and wherein the other of the heterozygous autosomal genes comprises one or more sequence variants of autosomal genes that are resistant to RNAi knock-down. In some embodiments, the autosomal gene is a haploinsufficient gene. In some embodiments, the haploinsufficient gene comprises VEGF, DLL4, or a combination thereof. In some embodiments, the haploinsufficient gene is VEGF. In some embodiments, the haploinsufficient gene is DLL4. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate.

### Single Sex Offspring in Poultry

In one aspect, the present disclosure provides methods and compositions utilizing RNAi and haploinsufficiency that knockdown an essential autosomal gene, e.g., haploinsufficient gene, to generate single sex offspring in poultry, e.g., chicken. In some instances, the single sex offspring is female offspring. For example, in chicken, the Z¹ that is from the female hen parent encodes component of RNAi knockdown machinery, e.g., siRNA or shRNA, targeting the essential autosomal gene, e.g., haploinsufficient gene. Further, both copies of the targeted autosomal genes of the engineered female chicken are modified to be resistant to RNAi knockdown, but express a wildtype protein. Alternatively, both copies of the targeted autosomal genes of the engineered female chicken have a sequence variant that makes them resistant to RNAi knockdown, but express a functional protein. In other words, the gene of interest, e.g., haploinsufficient gene, comprises a mutated sequence that cannot be knocked down by RNAi, but encodes a wildtype protein. In this instance, wildtype male chicken can be used. As a result of crossing between these two parents, male offspring express half dose of the essential gene, e.g., haploinsufficient gene, thereby making them unable to survive. This RNAi and haploinsufficiency that knockdown an essential autosomal gene, e.g., haploinsufficient gene, for generation of single sex offspring can be applied to other animal, all of which are compatible with methods of the present disclosure and contemplated herein.

In a different aspect, the present disclosure provides methods and compositions utilizing RNAi and haploinsufficiency that knockdown an essential autosomal gene, e.g., haploinsufficient gene, to generate single sex offspring in poultry, e.g. chicken. In some instances, the single sex offspring is male offspring. For example, in chicken, the W¹ that is from the female hen parent encodes component of RNAi knockdown machinery, e.g., siRNA or shRNA, targeting the essential autosomal gene, e.g., haploinsufficient gene. Further, both copies of the targeted autosomal genes of the engineered female chicken are modified to be resistant to RNAi knockdown, but express a wildtype protein. Alternatively, both copies of the targeted autosomal genes of the engineered female chicken have a sequence variant that makes them resistant to RNAi knockdown, but express a functional protein. In other words, the gene of interest, e.g., haploinsufficient gene, comprises a mutated sequence that cannot be knocked down by RNAi, but encodes a wildtype protein. In this instance, wildtype male chicken can be used. As a result of crossing between these two parents, male offspring express half dose of the essential gene, e.g., haploinsufficient gene, thereby unable to survive. This RNAi and haploinsufficiency that knockdown an essential autosomal gene, e.g., haploinsufficient gene, for generation of single sex offspring can be applied to other animals, all of which are compatible with methods of the present disclosure and contemplated herein.

### Single Sex Offspring in Mammals

In one aspect, the present disclosure provides methods and compositions utilizing RNAi and haploinsufficiency that knockdown an essential autosomal gene, e.g., haploinsufficient gene, to generate single sex offspring in animal such as cow. In some instances, the single sex offspring is female offspring. For example, in cow, the Y¹ that is from the male cow parent encodes component of RNAi knockdown machinery, e.g., siRNA or shRNA, targeting the essential autosomal gene, e.g., haploinsufficient gene. Further, both copies of the targeted autosomal genes of the engineered male cow are modified to be resistant to RNAi knockdown, but express a wildtype protein. Alternatively, both copies of the targeted autosomal genes of the engineered male cow have a sequence variant that makes them resistant to RNAi knockdown, but express a functional protein. In other words, the gene of interest, e.g., haploinsufficient gene, comprises a mutated sequence that cannot be knocked down by RNAi, but encodes a wildtype protein. In this instance, wildtype female cow can be used. As a result of crossing between these two parents, male offspring express half dose of the essential gene, e.g., haploinsufficient gene, thereby unable to survive. This RNAi and haploinsufficiency that knockdown an essential autosomal gene, e.g., haploinsufficient gene, for generation of single sex offspring can be applied to other animal, all of which are compatible with methods of the present disclosure and contemplated herein. Examples of animal include, but not limited to mammals, e.g., cow, mouse, rat, rabbit, guinea pig, bovine, chimpanzee, sheep, goat, and non-human primate.

In another aspect, the present disclosure provides methods and compositions utilizing RNAi and haploinsufficiency that knockdown an essential autosomal gene, e.g., haploinsufficient gene, to generate single sex offspring in animal such as cow. In some instances, the single sex offspring is male offspring. For example, in cow, the X¹ that is from the male cow parent encodes component of RNAi knockdown machinery, e.g., siRNA or shRNA, targeting the essential autosomal gene, e.g., haploinsufficient gene. Further, both copies of the targeted autosomal genes of the engineered male cow are modified to be resistant to RNAi knockdown, but express a wildtype protein. Alternatively, both copies of the targeted autosomal genes of the engineered male cow have a sequence variant that makes them resistant to RNAi knockdown, but express a functional protein. In other words, the gene of interest, e.g., haploinsufficient gene, comprises a mutated sequence that cannot be knocked down by RNAi, but encodes a wildtype protein. In this instance, wildtype female cow can be used. As a result of crossing between these two parents, female offspring express half dose of the essential gene, e.g., haploinsufficient gene, thereby unable to survive. This RNAi and haploinsufficiency that knockdown an essential autosomal gene, e.g., haploinsufficient gene, for generation of single sex offspring can be applied to other animal, all of which are compatible with methods of the present disclosure and contemplated herein. Examples of animal include, but not limited to mammals, e.g., cow, mouse, rat, rabbit, guinea pig, bovine, chimpanzee, sheep, goat, and non-human primate.

### Catalytically Inactive CRISPR and Haploinsufficiency to Generate Single Sex Offspring

CRISPR (clustered regularly interspaced short palindromic repeats)/Cas9 system provides a powerful method, not only for genome editing tools but also for gene regulation and epigenome editing. In genome editing tool, CRISPR/Cas9 system utilizes RNA-DNA base pairing of single guide RNA (sgRNA), which comprises target specific crRNA sequence and tracrRNA, to determine target specificity on the DNA. Once the specific sequence is identified, Cas9 endonuclease generates DNA double-strand breaks, which can be repaired by non-homology end joining (NHEJ) or homology-directed repair (HDR). NHEJ can result in random insertion/deletion, and in one aspect, this NHEJ can disrupt gene function at the target site, thus, providing a tool to generate knockouts of the target gene. In contrast, HDR can be utilized to insert a specific DNA template (either single stranded or double stranded) at the target site for precise gene editing (See Adil, M. et al. 2018. Nat Comm; Doundna, J. et al. 2014. Science).

Catalytically inactive CRISPR/Cas9 system has been developed for many applications including, but not limited to, gene regulation. In this regulation approach, which is also known as CRISPR interference (CRISPRi), catalytically inactive or dead Cas9 (dCas9) is mutated from the wildtype Cas9 to lack endonuclease activity. In this instance, when dCas9 is co-expressed with a sgRNA, they generate a DNA recognition complex that can specifically interfere with transcriptional process, thus, repressing expression of the targeted genes. CRISPRi can be used to repress expression of multiple target genes simultaneously and its effects are reversible (See Adil, M. et al. 2018, Nat Comm; Qi, L., et al. 2013. Cell.).

In some instances, the methods and compositions described herein utilize CRISPRi approach to generate single sex offspring in non-human animals. In some instances, the methods and compositions described herein utilize catalytically inactive or dead Cas9 (dCas9) to generate single sex offspring in non-human animals. In some instances, the dCas9 is co-expressed with sgRNA containing target sequence for the targeted gene via engineered vector. In some instances, the vector encoding the dCas9 and sgRNA is integrated into a chromosome. In some instances, the vector encoding the dCas9 and sgRNA is integrated into an allosome. In some instances, the vector encoding the dCas9 and sgRNA is integrated into an autosome.

In some instances, the targeted gene is a gene essential for development. In some instances, the targeted gene is haploinsufficient gene, which is lethal to the organism. Examples of haploinsufficient genes include, but are not limited to, vascular endothelial growth factor (VEGF) or delta-like 4 ligand (DLL4). In some embodiments, the haploinsufficient genes are described in, for example, Collins, R. et al. 2022. Cell Press, which is hereby incorporated by reference in its entirety.

### Poultry

In one aspect, instead of RNAi expression cassette, catalytically inactive CRISPR/Cas9 is integrated into the allosome Z¹. Genetic crossing is similar to RNAi and haploinsufficiency methods and compositions as described above. In this instance, the catalytically inactive CRISPR/Cas9 binds to the DNA of the haploinsufficient gene from the wildtype autosome A, thereby preventing the expression of the haploinsufficient gene. In this instance, the haploinsufficient gene on the mutated autosome A* comprises sequence variant to which the catalytically inactive CRISPR/Cas9 cannot bind. In some instances, the sequence variant is generated to have sequence mismatch from the catalytically inactive CRISPR/Cas9 guiding sequence.

### Mammals

In one aspect, instead of RNAi expression cassette catalytically inactive CRISPR/Cas9 is integrated into the allosome Y¹. Genetic crossing is similar to RNAi and haploinsufficiency methods and compositions as described above. In this instance, the catalytically inactive CRISPR/Cas9 binds to the DNA of the haploinsufficient gene from the wildtype autosome A, thereby preventing the expression of the haploinsufficient gene. In this instance, the haploinsufficient gene on the mutated autosome A* comprises sequence variant to which the catalytically inactive CRISPR/Cas9 cannot bind. In some instances, the sequence variant is generated to have sequence mismatch from the catalytically inactive CRISPR/Cas9 guiding sequence.

FIG. 1 depicts a genetic cross diagram showing how to generate single sex offspring using RNAi approach as described in the present disclosure. In chicken, Z and W are allosomes or sex chromosomes. ZW indicates female while ZZ indicates male. Z¹ indicates an allosome engineered to express one or more RNAi knockdown cassettes. These RNAi cassettes have the ability to reduce the expression of genes to which they have sequence identity. In some embodiments, the RNAi cassettes have sequence identity to one or more known genes known to be haploinsufficient genes, thus, are lethal to cells when the protein level is not expressed at the sufficient level. A and A* indicate an autosomal gene. The gene on A is wildtype and can be targeted by RNAi. However, the gene on A* comprises mutated that cannot be knocked down by RNAi, but encodes a wildtype protein.

In this genetic crossing, the female parent contributes a Z¹ allosome to the male offspring (genotype: AA* and Z¹Z), and that Z¹ allosome knocks down one or more haploinsufficient genes on the autosome(s) of the offspring on the A autosome such that development is suppressed. Therefore, the sex emerging from this cross are females (genotype: AA* and ZW).

In some instances, instead of RNAi knockdown cassettes, Z¹ is engineered to express a catalytically inactive CRISPR that binds to the RNA of haploinsufficient gene expressed from autosome A, and this prevent their translation. In this instance, the same gene on the autosome A* is a variant to which CRISPR cannot bind. Thus, the viable offspring are female.

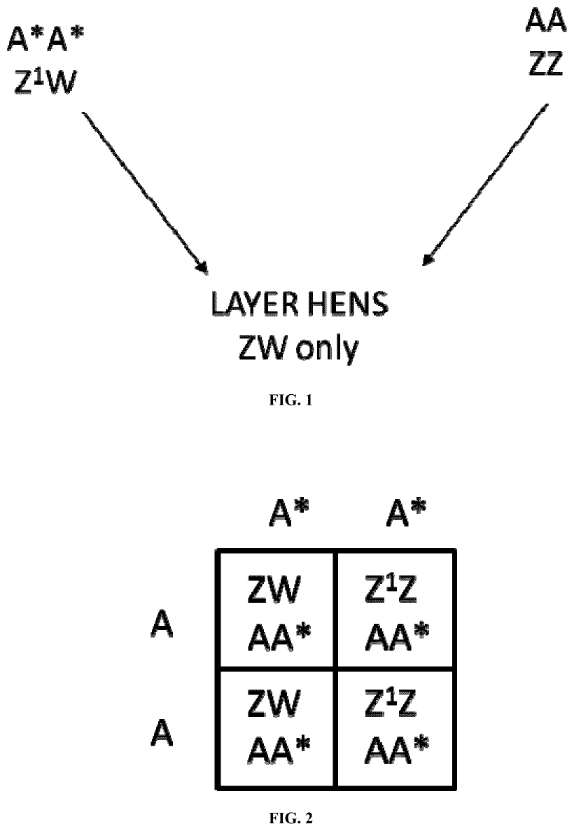

FIG. 2 depicts the Punnett Square of possible genotypic outcomes of offspring from genetic crossing of A*A* and Z¹W with AA and ZZ chicken. The female chicks (genotype: AA* and ZW) survive. The male chicks (genotype: AA* and Z¹Z) can express one copy of A protein because Z¹ express RNAi that knocks down protein from A* gene and is lethal due to haploinsufficiency.

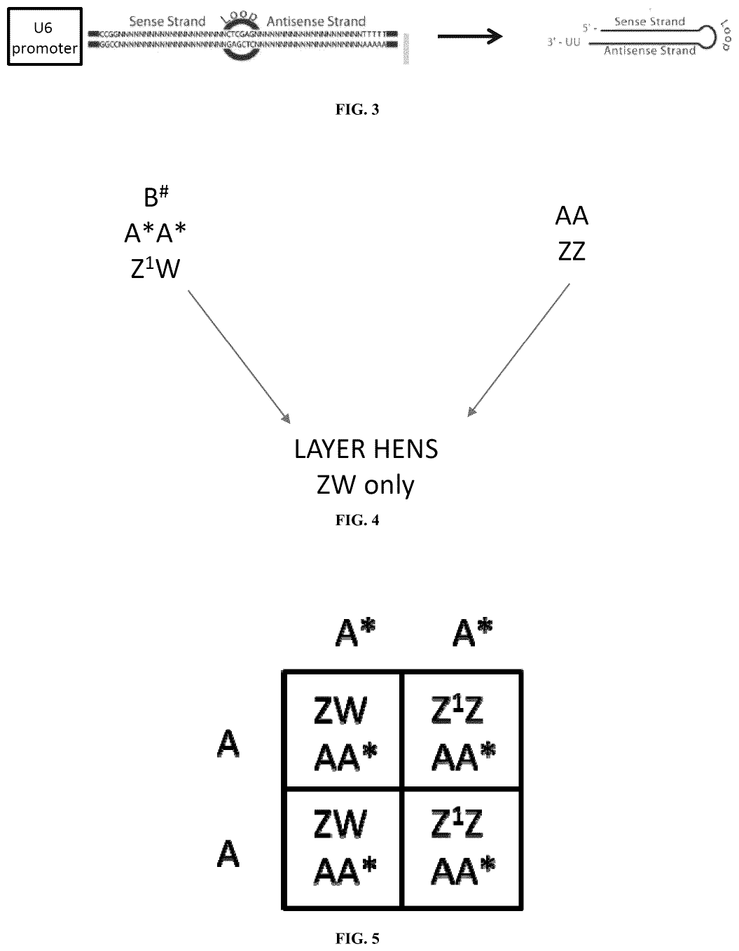

FIG. 3 depicts an example of an RNAi cassette integrated into the allosome. The RNAi cassette expresses short-hairpin RNA (shRNA) to knockdown the haploinsufficient gene on the autosome. The gene on wildtype A autosome comprises sequence variants that shRNA is targeted to bind and knockdown the expression of wildtype protein. However, the gene on A* comprises mutated that cannot be knocked down by RNAi, but encodes a wildtype protein. In this example, U6 promoter is utilized to drive the expression of shRNA from the RNAi cassette.

FIG. 4 depicts a genetic cross diagram showing how to generate single sex offspring using the RNAi approach with synthetic dose lethality. In chicken, Z and W are allosomes or sex chromosomes. ZW indicates a female while ZZ indicates a male. Z¹ indicates an allosome engineered to express one or more RNAi knockdown cassettes. These RNAi cassettes have the ability to reduce expression of genes to which they have sequence identity. In some embodiments, the RNAi cassettes have sequence identity to one or more known genes known to be haploinsufficient genes, thus are lethal to cells when the protein level is not expressed at the sufficient level. A, A*, and B# indicate autosomal genes. The gene on A is wildtype and can be targeted by RNAi. However, the gene on A* comprises mutated sequences transcribed into RNA that cannot be knocked down by RNAi but encode a wildtype protein. B# is an overexpression cassette that expresses high levels of a gene which shows synthetic dosage lethality with the haploinsufficiency of A.

In this genetic cross, the female parent contributes a Z¹ allosome to the male offspring (genotype AA* and Z¹Z) and that Z¹ allosome knocks down one or more haploinsufficient genes on the autosome(s) of the offspring on the A autosome such that development is suppressed. B# overexpresses a gene that has synthetic dosage lethality with A, ensuring the AA* Z¹Z males do not survive. Therefore the sex emerging from this cross are females (genotype AA* and ZW).

FIG. 5 depicts the Punnett Square of possible genotypic outcomes of offspring A*A* and Z¹W with AA and ZZ chicken. The female chicks (genotype: AA* and ZW) survive. The male chicks (genotype: AA* and Z¹Z) can express one copy of A protein because Z¹ express RNAi that knocks down protein from A* gene and is lethal due to haploinsufficiency.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: RNAi and Haploinsufficiency to Generate Single Sex Offspring

In this example, generation of single sex female layer hens is described by targeting genes for which haploinsufficiency is lethal using RNAi. As shown in FIG. 1, A and A* indicate an autosomal gene. Z¹ is a chromosome engineered to express one or more RNAi knockdown cassettes, in which an example is shown in FIG. 3. These RNAi knockdown cassettes have the ability to reduce the expression of genes to which they have sequence identity. In this case, the shRNA encoded by RNAi knockdown cassettes has sequence identity to one or more haploinsufficient genes. The genes on A* are sequence variants of the gene on A such that they are not knocked down by the shRNA but encodes a functional protein.

ZZ males have wildtype copies of this gene. ZW females have essential genes that contain sequence variants such that they are not amenable to the RNAi mediated knockdown. Thus, those embryos that receive a W chromosome from the hen will be wildtype for the haploinsufficient gene and thus alive. The live female offspring have mutations that are translated into a functional amino acid sequence in the protein.

As shown in FIG. 2, when the female parent contributes a Z chromosome to the offspring, that Z chromosome knocks down one or more haploinsufficient genes on the autosome(s) of the offspring such that development is suppressed. Therefore, the sex emerging from this cross are females.

FIG. 3 shows an example of RNAi knockdown cassette. For this method to work, RNAi sequence that silences the essential genes for which haploinsufficiency is lethal, e.g., VEGF gene or DLL4 gene, is first identified. The sequence is then cloned into the RNAi knockdown cassette as shown in FIG. 3. Next, the sequence of the host essential genes is mutated such that it is not susceptible to gene silencing by the RNAi knockdown cassette, but still express wildtype protein. In some instances, the mutation is made on the region of sequence that is not encoded for the protein so that the expression of wildtype protein still occur. Then the RNAi knockdown cassette is introduced onto the Z¹ allosome of a female hen. Also, this female hen is engineered to have mutated essential gene on both A* autosomes. As a result of breeding this engineered female hen with a wildtype male chicken, all of the offspring will receive one mutated essential gene (A*) and one wildtype (A) essential gene. Since all the male offspring will receive RNAi knockdown cassette from Z¹ allosome of the engineered female hen, the embryo will be functionally deficient in the protein and die because haploinsufficiency of these essential genes, e.g., VEGF or DDL4, is lethal. Thus, female offspring will be viable.

In some instances, Z¹ is further modified to overexpress a gene to make the haploinsufficiency based selection more stringent. In synthetic dosage lethality overexpression of the gene is unremarkable unless a different gene is under-expressed, in which case it is lethal. For example, VEGFR1 lacking a cytoplasmic domain is not lethal, acting as a possible VEGF sink. In combination with an RNAi that targets a single allele of VEGFR1, lethality of the VEGFR1 RNAi is increased and the stringency of sex selection is improved.

In some instances, instead of expressing shRNA from RNAi knockdown cassette, Z¹ expresses a catalytically inactive CRISPR/Cas9 that binds to the RNA for a haploinsufficient gene(s) expressed from chromosome A and prevents their translation. Similarly, the same gene on chromosome A* is a variant to which the catalytically inactive CRISPR/Cas9 cannot bind because of sequence mismatch. The male offspring dies. The viable offspring are female and not genetically modified.

### Example 2: RNAi Approach with Synthetic Dosage Lethality

In this case, Z¹ expresses RNAi for a gene essential for development. ZZ males have wildtype copies of this gene. ZW females have essential genes that contain sequence variants such that they are not amenable to the RNAi mediated knockdown. The gene in this example is VEGF, for which haploinsufficiency is lethal. A* is VEGF, which has a mutation transcribed to RNA but the resulting protein is wildtype. B# is a VEGFR1 expression cassette which increases the stringency of VEGF haploinsufficiency, optionally located on the Z chromosome. A is wildtype VEGF. Z¹ encodes a RNAi that targets wildtype VEGF. The result of a cross of a B# A*A* Z¹W female to a AA ZZ male results in only female progeny because only embryos that receive the W chromosome will have wildtype levels of VEGF because they have mutations in the VEGF locus that are translated into RNA that is not targeted by the RNAi. FIG. 4 is a diagram of the cross and FIG. 5 shows a Punnett square which shows the possible progeny from the cross.

1. A non-human vertebrate animal comprising one or more sequence variants of both autosomal genes, and an allosome modified to express one or more RNAi expression cassettes configured to reduce expression of the autosomal gene, wherein the one or more sequence variants of both autosomal genes do not have sequence identity with the one or more RNAi expression cassettes.

2. The non-human vertebrate animal of claim 1, wherein the RNAi expression cassettes are configured to reduce the expression of genes to which they have sequence identity.

3. The non-human vertebrate animal of claim 1, wherein the one or more sequence variants of both autosomal genes are resistant to RNAi knock-down.

4. The non-human vertebrate animal of claim 1, wherein the non-human vertebrate animal is selected from the group consisting of cow, mouse, rat, rabbit, guinea pig, chicken, fish, bird, reptile, camelid, bovine, chimpanzee, sheep, goat, and non-human primate.

5. The non-human vertebrate animal of claim 1, wherein the autosomal gene is a haploinsufficient gene.

6. The non-human vertebrate animal of claim 5, wherein the haploinsufficient gene comprises VEGF, DLL4, or combination thereof.

7.-8. (canceled)

9. The non-human vertebrate animal of claim 1, wherein the one or more RNAi expression cassettes comprise one or more RNAi sequences operatively linked to one promoter.

10.-28. (canceled)

29. A method of producing a single sex offspring of non-human vertebrate animals, the method comprising:
crossing (i) a first non-human vertebrate animal having a first genotype comprising one or more sequence variants of both autosomal genes, and an allosome modified to express one or more RNAi cassettes configured to reduce expression of the autosomal gene, wherein the one or more sequence variants of both autosomal genes do not have sequence identity with the one or more RNAi cassettes with (ii) a second non-human vertebrate animal having a second genotype comprising a wildtype genome with homozygous allosomes;
wherein a resulting progeny having a genotype comprising a heterozygous autosomal gene and the allosome modified to express one or more RNAi expression cassettes is not viable;
thereby creating a single sex offspring.

30. The method of claim 29, wherein the one or more RNAi expression cassettes reduce the expression of genes to which they have sequence identity.

31. The method of claim 29, wherein the autosomal gene is a haploinsufficient gene.

32. The method of claim 31, wherein the haploinsufficient gene comprises VEGF, DLL4, or a combination thereof.

33.-34. (canceled)

35. The method of claim 29, wherein the non-human vertebrate animal is selected from the group consisting of cow, mouse, rat, rabbit, guinea pig, chicken, fish, bird, reptile, camelid, bovine, chimpanzee, sheep, goat, and non-human primate.

36. The method of claim 29, wherein the one or more RNAi expression cassettes comprise one or more RNAi sequences operatively linked to one promoter.

37. The method of claim 29, wherein the allosome of the first non-human vertebrate animal is further modified to overexpress a gene.

38. A method of producing a single sex population of non-human vertebrate animals, comprising the steps of:
(a) obtaining (i) a first non-human vertebrate animal having a first genotype comprising one or more sequence variants of an autosomal gene, and an allosome integrated with an RNAi expression cassette comprising the following elements in 5′ to 3′ orientation: a promoter operatively linked thereto a nucleic acid sequence encoding for the expression of one or more RNAi cassettes, wherein the one or more RNAi expression cassettes are configured to reduce the expression of one or more autosomal genes;
(b) obtaining (ii) a second non-human vertebrate animal having wildtype genotype; and
(c) crossing the first non-human vertebrate animal and the second non-human vertebrate animal, wherein a resulting progeny comprising the allosome integrated with the RNAi expression cassette is not viable;
thereby creating a single sex offspring.

39. The method of claim 38, wherein the non-human vertebrate animal is selected from the group consisting of cow, mouse, rat, rabbit, guinea pig, chicken, fish, bird, reptile, camelid, bovine, chimpanzee, sheep, goat, and non-human primate.

40. The method of claim 38, wherein the autosomal gene is a haploinsufficient gene.

41. The method of claim 40, wherein the haploinsufficient gene comprises VEGF, DLL4, or a combination thereof.

42.-43. (canceled)

44. The method of claim 38, wherein the promoter is a U6 promoter or a H1 promoter.

45. The method of claim 38, wherein the allosome of the first non-human vertebrate animal is further modified to overexpress a gene.
